(19) United States
(12) Patent Application Publication
(10) Pub. No.: US 2008/0214505 A1
(43) Pub. Date: Sep. 4, 2008

(54) Risedronate compositions and their methods of use

(76) Inventors: David Ernest Burgio, Liberty Township, OH (US); Pamela Jean Schofield, Cincinnati, OH (US); Maurice Kent Gately, Morris Plains, NJ (US); Jun Shi, Livingston, NJ (US)

Correspondence Address: HOFFMANN-LA ROCHE INC.;PATENT LAW DEPARTMENT, 340 KINGSLAND STREET, NUTLEY, NJ, 07110, US

(21) Appl. No.: 12/077,623

(22) Filed: Mar. 20, 2008

Related U.S. Application Data
(63) Continuation of application No. 10/897,897, filed on Jul. 23, 2004.
Application No. 10/897,897 is a continuation-in-part of application No. 10/321,208, filed on Dec. 17, 2002, now abandoned.
(60) Provisional application No. 60/344,875, filed on Dec. 21, 2001.

(51) Int. Cl. A61K 31/675; A61P 19/00

(52) U.S. Cl. 514/89

(57) ABSTRACT

A method comprising orally administering to a human or other mammal a pharmaceutical composition comprising from about 65% to about 110% of the cumulative effective dose of risedronate or a pharmaceutically acceptable acid, salt, ester, solvate, or polymorph thereof according to a continuous dosing schedule of one, two, or three consecutive days per month is useful for treating or preventing osteoporosis and other bone metabolic disorders.

### CROSS REFERENCE

This application is a continuation-in-part of U.S. application Ser. No. 10/321,208, filed Dec. 17, 2002, which claims the benefit of U.S. Provisional Application No. 60/344,875, filed Dec. 21, 2001.

### FIELD OF THE INVENTION

The present invention relates to oral formulations of risedronate and their methods of use in the treatment and prevention of diseases related to bone remodeling or bone disorders such as, for example, osteoporosis. The methods of the present invention comprise administering to a human or other mammal in need thereof a pharmaceutical composition comprising from about 65% to about 110% of the cumulative effective dose of risedronate according to a continuous dosing schedule of one, two, or three consecutive days per month. The present invention also relates to pharmaceutical compositions of risedronate and kits for carrying out these methods.

### BACKGROUND OF THE INVENTION

The most common metabolic bone disorder is osteoporosis. Osteoporosis can be generally defined as the reduction in the quantity of bone, or the atrophy of skeletal tissue due to an imbalance in the normal resorption/formation cycle of bone within the bone remodeling unit. In general, there are two types of osteoporosis: primary and secondary. Secondary osteoporosis is the result of an identifiable disease process or agent. For example, glucocorticoid steroids are known to induce osteoporosis. See, for example, American College of Rheumatology Ad Hoc Committee on Glucocorticoid-Induced Osteoporosis, “Recommendations for the Prevention and Treatment of Glucocorticoid-Induced Osteoporosis,” Arthritis & Rheumatism, Vol. 44(7): 1496-1503 (July 2001); B. P. Lukert, M.D., F.A.C.P. “Glucocorticoid-Induced Osteoporosis,” Primer in the Metabolic Bone Diseases and Disorders of Mineral Metabolism, 4th Ed. 292-96, Publication of the American Society for Bone and Mineral Research, Murray J. Favus, M. D. Editor, Dept of Medicine, The University of Chicago Medical Center, Chicago, Ill. Approximately 85% of all osteoporosis is primary osteoporosis. See, for example, Matorie M. Luckey, M. D., “Evaluation of Postmenopausal Osteoporosis,” Primer on the Metabolic Bone Diseases and Disorders of Mineral Metabolism, 4th Ed. 273-77, Murray J. Favus, M. D. Editor, Dept of Medicine, The University of Chicago Medical Center, Chicago, Ill.; and “Osteoporosis Prevention, Diagnosis, and Therapy,” JAMA, Vol. 285(6): 785-95 (Feb. 14, 2001). Such primary osteoporosis includes postmenopausal osteoporosis, age-associated osteoporosis (affecting a majority of individuals over the age of 70 to 80) and idiopathic osteoporosis.

For some osteoporotic individuals, the loss of bone tissue is sufficiently great so as to cause mechanical failure of the bone structure. Bone fractures often occur, for example, in the hip and spine of women suffering from postmenopausal osteoporosis. Kyphosis (abnormally increased curvature of the thoracic spine) may also result. Although its etiology is not fully understood, there are many risk factors thought to be associated with osteoporosis. These include low body weight, low calcium intake, physical inactivity, and estrogen deficiency.

Many compositions and methods are described for the “treatment” of osteoporosis. Many of these include the use of bisphosphonates or other bone-active phosphonates. See, for examples, J. Y. Reginster et al., “Randomized Trial of the Effects of Risedronate on Vertebral Fractures in Women with Established Postmenopausal Osteoporosis,” Osteoporosis International, (2000) 11: 83-91; Steven T. Harris, MD et al., “Effects of Risedronate Treatment of Vertebral and Nonvertebral Fractures in Women With Postmenopausal Osteoporosis, A Randomized controlled Trial,” JAMA, Oct. 13, 1999, Vol. 282(14): 1344-52. Risedronate, or 1-hydroxy-2-(3-pyridinyl)-ethylidene-1,1-bisphosphonic acid, is a member of the class of compounds known as bisphosphonates. See U.S. Pat. No. 5,583,122, to Benedict et al., issued Dec. 10, 1996.

Continuous and cyclic administration of bisphosphonates alone or with other medicaments such as parathyroid hormone, calcium, and vitamin D have also been suggested as a therapy for osteoporosis. See, for example American College of Rheumatology Ad Hoc Committee on Glucocorticoid-Induced Osteoporosis, “Recommendations for the Prevention and Treatment of Glucocorticoid-Induced Osteoporosis,” Arthritis & Rheumatism, Vol. 44(7): 1496-1503 (July 2001); J. Y. Reginster, et al., “Randomized Trial of the Effects of Risedronate on Vertebral Fractures in Women with Established Postmenopausal Osteoporosis,” Osteoporosis International, 11: 83-91 (2000); Steven T. Harris, M D, et al., “Effects of Risedronate Treatment of Vertebral and Nonvertebral Fractures in Women With Postmenopausal Osteoporosis, A Randomized controlled Trial,” JAMA, Vol. 282 (14): 1344-52 (Oct. 13, 1999).

Adverse gastrointestinal effects have been associated with bisphosphonates as a class. Further, although calcium supplements are recommended for those at risk or suffering from osteoporosis, calcium-containing foods or supplements interfere with the absorption, and thus the efficacy, of bisphosphonates if taken simultaneously. To overcome these effects, patients taking bisphosphonates are instructed to take their medication with water and without food. Patients are further instructed to remain upright for thirty minutes after taking a bisphosphonate, and to take a calcium supplement at a different time of the day, or on a day when the patient is not taking a dose of a bisphosphonate.

These instructions can prove burdensome or difficult to remember for a patient who regularly takes a bisphosphonate. Thus, a less frequent dosing regimen would enhance patient convenience, which could lead to greater patient compliance with complicated treatment regimens associated with bisphosphonates.

Daily and weekly oral dosing of bisphosphonates is known in the art. See, for example, Harris, S. T. et al., “Two-year efficacy and tolerability of risedronate once a week for the treatment of women with postmenopausal osteoporosis,” Curr. Med. Res. Opin. 20(5): 757-64 (May 2004); Eisman, J. A. et al., “Upper gastrointestinal and overall tolerability of alendronate once weekly in patients with osteoporosis: results of a randomized, double-blind, placebo-controlled study,” Curr. Med. Res. Opin. 20(5): 699-705 (May 2004). Monthly oral dosing regimens have also been disclosed, however, current teachings indicate that greater than 100% of the cumulative effective dose of a bisphosphonate must be given in monthly treatment regimens in order to achieve comparable efficacy to that seen in daily or weekly dosing. For example, in U.S. Pat. Pub. 2003/0225039A1, by Bauss et al., published Dec. 4, 2003, the applicants teach that monthly oral treatment of “at least 120%, especially 120% to 200%, of the expected efficacious daily dose of a bisphosphonate offers incremental patient benefits with respect to convenience and compliance” (paragraph 0012). Bauss et al. further teach that this treatment regimen applies to risedronate (paragraph 0035). In U.S. Pat. Pub. 2003/0139378A1, by Daifotis et al., published Jul. 24, 2003, the applicants teach intermittent dosing of a “relatively high unit dose” of a bisphosphonate. For example, Daifotis et al. teach a once monthly liquid oral dose useful for the treatment of osteoporosis comprising from about 280 mg to about 560 mg of alendronate, on an alendronic acid active weight basis (paragraph 0115).

Unlike these teachings, Applicants have found that delivering from about 65% to about 110% of the cumulative effective dose of risedronate according to a continuous dosing schedule of one, two, or three consecutive days per month provides comparable efficacy as that seen with daily or weekly oral dosing of risedronate. Such intermittent dosing regimens can increase patient satisfaction, thus leading to increased patient compliance with prescribed risedronate therapies.

### SUMMARY OF THE INVENTION

The present invention relates to a method for treating or preventing a bone disorder in a human or other mammal in need thereof comprising orally administering to said mammal a pharmaceutical composition comprising from about 65% to about 110% of the cumulative effective dose of risedronate or a pharmaceutically acceptable acid, salt, ester, solvate, or polymorph thereof according to a continuous dosing schedule of one, two, or three consecutive days per month. The invention further relates to pharmaceutical compositions and kits suitable for use with the methods of the present invention.

### DETAILED DESCRIPTION OF THE INVENTION

Risedronate, or 1-hydroxy-2-(3-pyridinyl)-ethylidene-1,1-bisphosphonic acid, is a member of the class of compounds known as bisphosphonates. See U.S. Pat. No. 5,583,122, to Benedict et al., issued Dec. 10, 1996. Risedronate has the chemical structure:

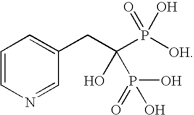

The term “risedronate,” as used herein, is understood to include any pharmacologically active form of risedronate including, but not limited to, pharmaceutically acceptable acids, salts, esters, solvates, or polymorphs thereof. In one embodiment of the invention, the sodium salt form is selected from the group consisting of hemipentahydrate, monohydrate, and mixtures thereof. Reference to a specific weight or percentage of risedronate in the present invention is on an anhydrous monosodium salt basis, unless otherwise indicated herein. For example, a pharmaceutical composition that “comprises about 150 mg risedronate” contains the equivalent of about 150 mg risedronate anhydrous monosodium salt.

One embodiment of the invention comprises a method for treating or preventing a bone disorder in a mammal in need thereof comprising orally administering to said mammal a pharmaceutical composition comprising from about 65% to about 110% of the cumulative effective dose of risedronate or a pharmaceutically acceptable acid, salt, ester, solvate, or polymorph thereof according to a continuous dosing schedule of one, two, or three consecutive days per month. In another embodiment of the invention, the pharmaceutical composition comprises from about 65% to about 100% of the cumulative effective dose of risedronate. In still another embodiment of the invention, the pharmaceutical composition comprises about 100% of the cumulative effective dose of risedronate.

A “pharmaceutically-acceptable salt” is a cationic salt formed at any acidic (e.g., hydroxamic or carboxylic acid) group, or an anionic salt formed at any basic (e.g., amino) group. Many such salts are known in the art, as described in WO 87/05297, by Johnston et al., published Sep. 11, 1987. Preferred cationic salts include the alkali metal salts (such as sodium and potassium), and alkaline earth metal salts (such as magnesium and calcium) and organic salts. Preferred anionic salts include the halides (such as chloride salts), sulfonates, carboxylates, phosphates, and the like.

The term “pharmaceutically acceptable ester,” as used herein, refers to an ester of risedronate that does not interfere with the bone resorption inhibition activity of risedronate or that is readily converted by an animal to yield risedronate.

The term “solvate,” as used herein, refers to a compound formed by the chemical combination of a solvent and another substance in a specific molecular ratio. In one embodiment of the invention, the solvent is water and the resulting solvate is a hydrate. Non-limiting examples of pharmaceutically acceptable solvates of risedronate include the hemipentahydrate and monohydrate forms, as described in U.S. Pat. No. 6,410,520, to Cazer et al., issued Jun. 25, 2002.

The term “polymorph,” as used herein, refers to the existence of a substance in an alternate form having different physical and/or chemical properties. Non-limiting examples of polymorphs of risedronate include crystal form variations such as plates and as needles.

Such salts, esters, solvates, and polymorphs are well understood by the skilled artisan, and the skilled artisan is able to prepare any number of these given the knowledge in the art. Furthermore, it is recognized that the skilled artisan may prefer one salt, ester, solvate, or polymorph over another for reasons of solubility, stability, formulation ease, and the like. Determination and optimization of such salts, esters, solvates, and polymorphs is within the purview of the skilled artisan's practice.

The terms “continuous” and “continuously,” as used herein, mean at regular specified intervals. For example, a continuous frequency of once a month means that the active is given once a month for an unspecified period of time or for as long as treatment is necessary.

The term “month” is used in accordance with the generally accepted meaning as a measure of time amounting to approximately four weeks, approximately 30 days, or approximately 1/12 of a calendar year.

The terms “once a month,” “monthly,” or “once monthly,” as used herein, mean that a unit dose is administered once, i.e., one time, during a monthly period. Non-limiting examples of once monthly schedules include the following: (a) a unit dose is administered once daily the first day of each calendar month; (b) a unit dose is administered once daily every four weeks; and (c) a unit dose is administered once daily the first day of every 30-day period. In one embodiment of the invention, each unit dose of risedronate suitable for administration on a once monthly regimen comprises from about 97.5 to about 165 mg risedronate. In another embodiment of the invention, each unit dose suitable for administration on a once monthly regimen comprises from about 100 to about 150 mg risedronate. In yet another embodiment of the invention, each unit dose suitable for administration on a once monthly regimen comprises about 150 mg risedronate.

The terms “twice a month” or “twice monthly” mean that a unit dose is administered twice, i.e., two times, during a monthly period. In a twice monthly regimen, the unit doses are administered on two consecutive days. Non-limiting examples of twice monthly schedules include the following: (a) a unit dose is administered once daily the first two days of a calendar month; (b) a unit dose is administered the last day of one calendar month and the first day of the following calendar month; (c) a unit dose is administered once daily the first two days of every four week period; and (d) a unit dose is administered once daily the first two days of every 30-day period. In one embodiment of the invention, each unit dose of risedronate suitable for administration on a twice monthly regimen comprises from about 48.75 to about 82.5 mg risedronate. In another embodiment of the invention, each unit dose suitable for administration on a twice monthly regimen comprises from about 50 to about 75 mg risedronate. In yet another embodiment of the invention, each unit dose suitable for administration on a twice monthly regimen comprises about 75 mg risedronate.

The terms “three times a month” or “thrice monthly” mean that a unit dose is administered thrice, i.e., three times, during a monthly period. In a thrice monthly schedule, the unit doses are administered on three consecutive days. Non-limiting examples of thrice monthly schedules include the following: (a) a unit dose is administered each day for the first three days of a calendar month; (b) a unit dose is administered the last day of one calendar month and each of the first two days of the following calendar month; (c) a unit dose is administered once daily the first three days of every four week period; and (d) a unit dose is administered once daily the first three days of every 30 day period. In one embodiment of the invention, each unit dose of risedronate suitable for administration on a thrice monthly regimen comprises from about 32.5 to about 55 mg risedronate. In another embodiment of the invention, each unit dose suitable for administration on a thrice monthly regimen comprises from about 33 to about 50 mg risedronate. In yet another embodiment of the invention, each unit dose suitable for administration on a thrice monthly regimen comprises about 50 mg risedronate.

The term “unit dose” or “unit dosage” means one or more dosage forms containing an amount of pharmaceutical active or nutrient suitable for administration in one single dose, according to sound medical practice. The present invention is particularly useful for the administration of unit doses in the form of tablets and capsules.

The term “cumulative effective dose” means the effective daily dose multiplied by the approximate number of days in the treatment period. For example, if a bisphosphonate is dosed at a level of 5 mg per day, the cumulative effective dose for a seven day period is (5 mg)×(7 days), or 35 mg. The cumulative effective dose for a monthly period is (5 mg)×(30 days), or 150 mg.

The term “combined unit dose of calcium and vitamin D,” as used herein, means a single unit dose comprising both calcium and vitamin D.

The term “IU,” as used herein, means International Units. One microgram of vitamin D is approximately 40 International Units.

The term “nutrient,” as used herein, means any nutritional or dietary supplement including but not limited to vitamins, minerals, amino acids, herbs or other botanicals, or concentrates, metabolites, constituents, extracts, or combinations of the same.

The preferred nutrients to be administered in the bisphosphonate treatment regimen are calcium and/or vitamin D. Oral forms of calcium suitable for use in the present invention include capsules, compressed tablets, chewable tablets, and the like. Typical salt forms of calcium suitable for use in the present invention include but are not limited to calcium carbonate, calcium citrate, calcium malate, calcium citrate malate, calcium glubionate, calcium gluceptate, calcium gluconate, calcium lactate, dibasic calcium phosphate, and tribasic calcium phosphate. In one embodiment of the invention, calcium can be administered at doses of 400 mg to 1500 mg of calcium per day. In another embodiment of the invention, calcium can be administered at doses of 400 mg to 1500 mg of calcium per day, on the days in between the days when the patient takes a unit dose of pharmaceutical active. If a calcium supplement and risedronate are dosed on the same day, the patient should take the bisphosphonate and the nutrient at different times of the day. For example, the patient may take a unit dose of risedronate in the morning, and a calcium supplement 4 hours later.

The term “vitamin D,” as used herein, refers to any form of vitamin D that may be administered to a mammal as a nutrient. Vitamin D is metabolized in the body to provide what is often referred to as “activated” forms of vitamin D. The term “vitamin D” can include activated and non-activated forms of vitamin D, as well as precursors and metabolites of such forms. Precursors of these activated forms include vitamin D₂ (ergocalciferol, produced in plants) and vitamin D₃ (cholecalciferol, produced in skin and found in animal sources and used to fortify foods). Vitamins D₂ and D₃ have similar biological efficacy in humans. Non-activated metabolites of vitamins D₂ and D₃ include hydroxylated forms of vitamins D₂ and D₃. Activated vitamin D analogs cannot be administered in large doses on an intermittent schedule, due to their toxicity in mammals. However, non-activated vitamin D₂, vitamin D₃, and their metabolites may be administered in larger doses than “active” forms of vitamin D on an intermittent basis, without toxicity. In one embodiment of the invention, vitamin D can be administered at doses of 100 IU to 10,000 IU of vitamin D per day. In another embodiment of the invention, vitamin D can be administered at doses of 100 IU to 10,000 IU of vitamin D per day, on the days in between the days when the patient takes a unit dose of risedronate.

In another embodiment of the invention, the nutrient is a unit dose comprising both calcium and vitamin D. In one embodiment, the unit dose comprises about 500 mg calcium and about 400 IU to about 440 IU vitamin D, to be administered daily. In a further embodiment, the unit dose comprises about 500 mg calcium and about 400 IU to about 440 IU vitamin D, to be administered on the days in between the days when the patient takes the unit dose of risedronate. If a calcium-containing supplement and risedronate are dosed on the same day, the patient should take the bisphosphonate and the nutrient at different times of the day. For example, the patient may take a unit dose of risedronate in the morning, and a calcium-containing supplement 4 hours later.

### Pharmaceutical Compositions

The present invention further relates to a pharmaceutical composition suitable for administration according to a continuous dosing schedule of one, two, or three consecutive days per month, said pharmaceutical composition comprising:

(a) from about 65% to about 110% of the cumulative effective dose of risedronate; and

(b) one or more pharmaceutically-acceptable excipients.

The term “pharmaceutically-acceptable excipient,” as used herein, means any physiologically inert, pharmacologically inactive material known to one skilled in the art, which is compatible with the physical and chemical characteristics of risedronate. Pharmaceutically-acceptable excipients include, but are not limited to, polymers, resins, plasticizers, fillers, lubricants, diluents, binders, disintegrants, solvents, co-solvents, surfactants, preservatives, sweetening agents, flavoring agents, pharmaceutical grade dyes or pigments, and viscosity agents.

Flavoring agents and dyes and pigments among those useful herein include those described in Handbook of Pharmaceutical Excipients (4th ed., Pharmaceutical Press 2003).

Suitable co-solvents include, but are not limited to, ethanol, isopropanol, and acetone.

Suitable surfactants include, but are not limited to, polyoxyethylene sorbitan fatty acid esters, polyoxyethylene monoalkyl ethers, sucrose monoesters, sodium lauryl sulfate, Tween 80®, and lanolin esters and ethers.

Suitable preservatives include, but are not limited to, phenol, alkyl esters of parahydroxybenzoic acid, benzoic acid and the salts thereof, boric acid and the salts thereof, sorbic acid and the salts thereof, chlorbutanol, benzyl alcohol, thimerosal, phenylmercuric acetate and nitrate, nitromersol, benzalkonium chloride, cetylpyridinium chloride, methyl paraben, and propyl paraben.

Suitable fillers include, but are not limited to, starch, lactose, sucrose, maltodextrin, and microcrystalline cellulose.

Suitable plasticizers include, but are not limited to, triethyl citrate, polyethylene glycol, propylene glycol, dibutyl phthalate, castor oil, acetylated monoglycerides, and triacetin.

Suitable polymers include, but are not limited to, ethylcellulose, cellulose acetate trimellitate, hydroxypropylmethylcellulose phthalate, cellulose acetate phthalate, polyvinyl acetate phthalate, and Eudragit® L 30-D, Eudragit® L 100-55, and Eudragit® S 100 (Röhm Pharma GmbH and Co. KG, Dammstadt, Germany), and Acryl-EZE® and Sureteric®g (Colorcon, Inc., West Point, Pa.).

Suitable lubricants include, but are not limited to, magnesium stearate, stearic acid, and talc.

The pharmaceutical compositions of the present invention may optionally comprise a chelating agent. The term “chelating agent,” as used herein, means a molecule containing two or more electron donor atoms that can form coordinate bonds to a single metal ion. The term “chelating agent” is understood to include the chelating agent as well as salts thereof. For example, the term “chelating agent” includes citric acid as well as its salt forms.

The most common and widely used chelating agents coordinate to metal atoms through oxygen or nitrogen donor atoms, or both. Other less common chelating agents coordinate through sulfur in the form of —SH (thiol or mercapto) groups. After the first coordinate bond is formed, each successive donor atom that binds creates a ring containing the metal atom. A chelating agent may be bidentate, tridentate, tetradentate, etc., depending upon whether it contains two, three, four, or more donor atoms capable of binding to the metal atom. See Kirk-Othmer Encyclopedia of Chemical Technology (4th ed. 2001).

Chelating agents suitable for use in the present invention include any pharmaceutically-acceptable chelating agent. Non-limiting examples of chelating agents suitable for use in the present invention include EDTA, citric acid, malic acid, tartaric acid, lactic acid, aspartic acid, glutamic acid, lysine, sodium hexametaphosphate, and combinations thereof. In one embodiment of the present invention, the chelating agent is EDTA, citric acid, or sodium hexametaphosphate.

In another embodiment of the invention, a monodentate complexing agent may be used in place of a polydentate chelating agent. Suitable monodentate complexing agents include, but are not limited to, phosphates (e.g., sodium phosphate, sodium aluminum phosphate, sodium acid phosphate, dipotassium phosphate, disodium phosphate, monobasic) and carboxylic acids (e.g., fumaric acid, acetic acid). A preferred monodentate complexing agent is acetic acid.

The amount of chelating agent present in the oral dosage form of the present invention will depend on the particular chelating agent selected and the amount of bisphosphonate active ingredient present in the oral dosage form. Generally, the oral dosage forms of the present invention will contain a safe and effective amount of a chelating agent suitable for achieving the desired chelating effect. In one embodiment, the oral dosage form contains from about 10 mg to about 1000 mg of a chelating agent per unit dose. In another embodiment, the oral dosage forms contain from about 10 mg to about 500 mg of a chelating agent per unit dose. When the chelating agent is EDTA, the preferred range is from about 10 mg to about 500 mg, preferably from about 25 mg to about 250 mg per unit dose. When the chelating agent is citric acid or any other chelating agent, the preferred range is from about 25 mg to about 1000 mg, preferably from about 50 mg to about 500 mg per unit dose.

The pharmaceutical compositions of the present invention may optionally comprise a film coating or an enteric coating. Excipients suitable for use in a film coating include, but are not limited to, hydroxypropylmethylcellulose, hydroxypropylcellulose, gelatin, polyvinylpyrrolidone, lactose, polyethylene glycol, talc, microcrystalline cellulose, and polyvinyl alcohol. Excipients suitable for use in an enteric coating include, but are not limited to, cellulose acetate trimellitate, hydroxypropylmethylcellulose phthalate, cellulose acetate phthalate, polyvinyl acetate phthalate, Eudragit® L 30-D, Eudragit® L 100-55, Eudragit® S 100 (Röhm Pharma GmbH and Co. KG, Darmstadt, Germany), Acryl-EZE® and Sureteric®& (Colorcon, Inc., West Point, Pa.), triethyl citrate, polyethylene glycol, propylene glycol, dibutyl phthalate, castor oil, acetylated monoglycerides, triacetin, and talc.

### Kits

The kits of the present invention are particularly useful for administering risedronate according to a continuous dosing schedule of one, two, or three consecutive days per month. Such kits comprise one or more unit doses of risedronate and a means for facilitating compliance with methods of this invention. The kits of the invention provide a convenient and effective means for assuring that the subject to be treated takes the appropriate active in the correct dosage in the correct manner. The compliance means of such kits includes any means which facilitates administering the actives according to a method of this invention. Such compliance means includes instructions, packaging, and dispensing means, and combinations thereof. The kits can also comprise a means for aiding the memory, including but not limited to a listing of the days of the week, numbering, illustrations, arrows, Braille, calendar stickers, reminder cards, or other means specifically selected by the patient. Examples of packaging and dispensing means are well known in the art, including those described in U.S. Pat. No. 4,761,406, Flora et al., issued Aug. 2, 1988; and U.S. Pat. No. 4,812,311, Uchtman, issued Mar. 14, 1989. Examples of particular arrangements of unit doses include those described in U.S. patent application Ser. No. 10/789,525, by Cawthray et al., filed Feb. 27, 2004.

Optionally, the kits can comprise at least one unit dose of a risedronate and at least one unit dose of an accompanying nutrient.

The following are non-limiting examples of embodiments of the present invention.

### EXAMPLES

### Example 1

A 65 kg woman diagnosed with postmenopausal osteoporosis is prescribed a pharmaceutical composition comprising 150 mg risedronate, to be taken once monthly. The patient takes the oral dosage form the first day of each calendar month. A biopsy of iliac crest bone is taken at two years and reveals an increase in mean wall thickness of the remodeling units compared to her baseline biopsy.

### Example 2

A 70 kg man diagnosed with osteoporosis is prescribed a pharmaceutical composition comprising 125 mg risedronate, to be taken once monthly. The patient takes the oral dosage form the last day of each calendar month. A biopsy of iliac crest bone is taken at two years and reveals an increase in mean wall thickness of the remodeling units compared to his baseline biopsy.

### Example 3

A 62 kg woman diagnosed with postmenopausal osteoporosis is prescribed a pharmaceutical composition to be taken twice monthly. Each unit dose of the pharmaceutical composition comprises 75 mg risedronate. The patient takes a unit dose of the pharmaceutical composition once per day on the Saturday and Sunday of the first weekend of each calendar month. A biopsy of iliac crest bone is taken at two years and reveals an increase in mean wall thickness of the remodeling units compared to her baseline biopsy.

### Example 4

A 72 kg man diagnosed with osteoporosis is prescribed a pharmaceutical composition to be taken thrice monthly. Each unit dose of the pharmaceutical composition comprises 50 mg risedronate. The patient takes a unit dose of the pharmaceutical composition once per day on the Friday, Saturday, and Sunday of the first weekend of each calendar month. A biopsy of iliac crest bone is taken at two years and reveals an increase in mean wall thickness of the remodeling units compared to his baseline biopsy.

### Example 5

An open label, multicenter, randomized, parallel group study is performed to compare lumbar spine bone mineral density (LSBMD) in participants taking 50 mg risedronate each day for three consecutive days per month for six months with participants taking 5 mg risedronate daily for six months. LSBMD is measured by dual energy X-ray absorptiometry (DXA) at baseline and 6 months after treatment. At the end of the treatment period, participants taking 50 mg risedronate each day for three consecutive days per month show an increase in LSBMD as compared to baseline LSBMD measurements. Further, the increase in LSBMD recorded for participants taking 50 mg risedronate each day for three consecutive days per month is comparable to that recorded for participants taking 5 mg risedronate daily for six months.

### Example 6

A blinded, multicenter, randomized, parallel group study is performed to compare lumbar spine bone mineral density (LSBMD) in participants taking 100 mg, 150 mg, or 200 mg risedronate one day per month for six months with participants taking 5 mg risedronate daily for six months. LSBMD is measured by dual energy X-ray absorptiometry (DXA) at baseline and 6 months after treatment. At the end of the treatment period, participants taking 100 mg, 150 mg, or 200 mg doses of risedronate once monthly show an increase in LSBMD as compared to baseline LSBMD measurements. Further, the increase in LSBMD recorded for participants taking 100 mg, 150 mg, or 200 mg doses of risedronate once monthly for six months is comparable to that recorded for participants taking 5 mg risedronate daily for six months.

### Example 7

A blinded, multicenter, randomized, parallel group study is performed to compare lumbar spine bone mineral density (LSBMD) in participants taking 100 mg, 150 mg, or 200 mg risedronate one day per month for one year with participants taking 5 mg risedronate daily for one year. LSBMD is measured by dual energy X-ray absorptiometry (DXA) at baseline and one year after treatment. At the end of the treatment period, participants taking 100 mg, 150 mg, or 200 mg doses of risedronate once monthly show an increase in LSBMD as compared to baseline LSBMD measurements. Further, the increase in LSBMD recorded for participants taking 100 mg, 150 mg, or 200 mg doses of risedronate once monthly for one year is comparable to that recorded for participants taking 5 mg risedronate daily for one year.

All documents cited are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. A method for treating or inhibiting osteoporosis comprising commencing treatment by orally administering to a subject in need of such treatment, a first dose, on a single day, of a pharmaceutical composition comprising from about 100 mg to about 150 mg of risedronate or an amount of a pharmaceutically acceptable salt thereof that is equivalent to about 100 mg to about 150 mg of risedronate and continuing said treatment by orally administering, once monthly on a single day, a pharmaceutical composition comprising from about 100 mg to about 150 mg of risedronate or an amount of a pharmaceutically acceptable salt thereof that is equivalent to from about 100 mg to about 150 mg of risedronate.

19. The method according to claim 18, wherein the pharmaceutical composition comprises about 100 mg of risedronate or an amount of a pharmaceutically acceptable salt thereof that is equivalent to about 100 mg of risedronate.

20. The method according to claim 18, wherein the pharmaceutical composition comprises about 150 mg of risedronate or an amount of a pharmaceutically acceptable salt thereof that is equivalent to about 150 mg of risedronate.

21. The method of claim 18, wherein the pharmaceutical composition comprises 100 mg of risedronate or an amount of a pharmaceutically acceptable salt thereof that is equivalent to 100 mg of risedronate.

22. The method of claim 18 wherein the pharmaceutical composition comprises 150 mg of risedronate or an amount of a pharmaceutically acceptable salt thereof that is equivalent to 150 mg of risedronate.

23. The method of claim 18 wherein said pharmaceutical composition is a solid pharmaceutical composition.

24. The method of claim 20 wherein said pharmaceutical composition is a solid pharmaceutical composition.

25. The method of claim 22 wherein said pharmaceutical composition is a solid pharmaceutical composition.

26. A method for treating or inhibiting osteoporosis consisting of orally administering to a subject in need of such treatment, once monthly, a pharmaceutical composition comprising from about 100 mg to about 150 mg of risedronateor an amount of a pharmaceutically acceptable salt thereof that is equivalent to about 100 mg to about 150 mg of risedronate.

27. The method according to claim 26, wherein the pharmaceutical composition comprises about 100 mg of risedronate or an amount of a pharmaceutically acceptable salt thereof that is equivalent to about 100 mg of risedronate.

28. The method according to claim 26, wherein the pharmaceutical composition comprises about 150 mg of risedronate or an amount of a pharmaceutically acceptable salt thereof that is equivalent to about 150 mg of risedronate.

29. The method of claim 26 wherein the pharmaceutical composition comprises 100 mg of risedronate or an amount of a pharmaceutically acceptable salt thereof that is equivalent to 100 mg of risedronate.

30. The method of claim 26 wherein the pharmaceutical composition comprises 150 mg of risedronate or an amount of a pharmaceutically acceptable salt thereof that is equivalent to 150 mg of risedronate.

31. The method of claim 26 wherein said pharmaceutical composition is a solid pharmaceutical composition.

32. The method of claim 28 wherein said pharmaceutical composition is a solid pharmaceutical composition.

33. The method of claim 30 wherein said pharmaceutical composition is a solid pharmaceutical composition.
